(19) United States
(12) Patent Application Publication
(10) Pub. No.: US 2009/0118316 A1
(43) Pub. Date: May 7, 2009

(54) Methods for Augmenting Bone

(75) Inventors: Lisa M. Olson, Hopkinton, MA (US); Thomas A. Brown, Mystic, CT (US); Leonard Buckbinder, Pawcatuck, CT (US); Angel Guzman-Perez, Mystic, CT (US); John C. Kath, La Mesa, CA (US); Hua Zhu Ke, Newbury Park, CA (US); Michael J. Luzzio, Noank, CT (US)

Correspondence Address: PFIZER INC., PATENT DEPARTMENT, MS8260-1611, EASTERN POINT ROAD, GROTON, CT, 06340, US

(73) Assignee: PFZER INC.

(21) Appl. No.: 11/570,815

(22) PCT Filed: Jun. 10, 2005
(86) PCT No.: PCT/IB2005/002127

§ 371 Date: Dec. 18, 2006

Related U.S. Application Data
(60) Provisional application No. 60/581,407, filed on Jun. 21, 2004.

(51) Int. Cl. A61K 31/506; A61K 31/405; A61K 31/40

(52) U.S. Cl. 514/275; 514/414; 514/408

(57) ABSTRACT

The present invention relates to methods of stimulating osteoblast function with a PYK2 inhibitor in subjects with osteoporosis, bone fractures, non-unions, pseudoarthroses, periodontal disease or other disorders of bone metabolism. Optionally, the method further comprises administration of a second therapeutic bone agent. The present invention also relates to methods to identify a PYK2 inhibitor effective as a therapeutic bone agent comprising administering a test agent to an osteoblast-like cell and determining if osteoblast function is stimulated. Optionally, the identifying method further comprises contacting the test agent with PYK2 and determining if PYK2 activity is inhibited.

### FIELD OF THE INVENTION

The present invention relates to methods of treatment for subjects with osteoporosis, bone fractures, non-unions, pseudoarthroses, periodontal disease and other disorders of bone metabolism. The present invention also related to assays to identify therapeutic agents useful for stimulating an osteoblast function.

### BACKGROUND OF INVENTION

Bone is a dynamic organ, which undergoes growth, remodeling, and repair (i.e. repetitive cycles of formation and resorption). The development and maintenance of the skeleton requires the coordinated activities of bone-forming osteoblasts and bone-resorbing osteoclasts. When resorption exceeds formation, there will be a loss of bone mass (osteopenia) and/or bone integrity (osteoporosis).

Whereas bone loss is a progressive phenomenon, which begins, in early adult life, it rapidly accelerates in women at time of menopause (natural or surgical) and such loss is greatest within two years of estrogen deprivation. During this accelerated phase, bone formation is greatly reduced. It should also be noted that bone resorption also decreases, but to a lesser extent.

Pharmaceutical agents that decrease bone resorption (“antiresorptives”) or that increase bone formation (bone anabolics) have been the targets for new therapies. Nonetheless, the therapeutic efficacy of such agents is limited by the fact osteoblast and osteoclast function is tightly coupled—agents that stimulate osteoblasts can stimulate osteoclasts (and vice versa) and inhibition of one can similarly inhibit the other.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 520% of patients dying within one year, and over 50% of survivors being physically impaired.

The elderly are at greatest risk of osteoporosis, and the problem is therefore predicted to increase significantly with the aging of the population. Worldwide fracture incidence is forecasted to increase three-fold over the next 60 years, and one study estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

In addition to osteoporosis, approximately 20-25 million women and an increasing number of men have detectable vertebral fractures as a consequence of reduced bone mass, with an additional 250,000 hip fractures reported yearly in America alone. The latter case is associated with a 12% mortality rate within the first two years and with a 30% rate of patients requiring nursing home care after the fracture. While this is already significant, the economic and medical consequences of convalescence due to slow or imperfect healing of these bone fractures is expected to increase, due to the aging of the general population. While there are several promising therapies (bisphosphonates, etc.) in development to prevent bone loss with age and thus reduce the probability of incurring debilitating fractures, these therapies are not indicated for restoration of bone mass once the fracture has occurred.

An imbalance of bone formation and bone resorption can also occur in localized regions of the skeleton, even in subjects with normal total bone density. For example, local bone erosion and systemic bone loss are hallmarks of rheumatoid arthritis and cause progressive disability.

During bone fracture repair, when diminished levels of bone formation are accompanied with a more robust bone resorption, delayed healing may be clinically significant.

Estrogens have been shown (Bolander et al., 38th Annual Meeting Orthopedic Research Society, 1992) to improve the quality of the healing of appendicular fractures. Therefore, estrogen replacement therapy might appear to be a method for the treatment of fracture repair. However, patient compliance with estrogen therapy is relatively poor due to its side effects, including the resumption of menses, mastodynia, an increased risk of uterine cancer, an increased perceived risk of breast cancer, and the concomitant use of progestins. In addition, men are likely to object to the use of estrogen treatment. Clearly the need exists for a therapy which would be beneficial to patients who have suffered debilitating bone fractures or who have low bone mass and which would increase patient compliance.

The proline-rich tyrosine kinase (PYK2, also known as CAKβ and RAFTK) is a member of the FAK (focal adhesion kinase) family. PYK2 is expressed in neuronal and hemopoietic cells, and recently was shown to be highly expressed in osteoclasts (Lakkakorpi et al., J Biol. Chem. 2003 Mar. 28; 278(13):11502-12.

Further, it has been hypothesized that PYK2 plays a key role in the Src-dependent regulation of the adhesion and motility of osteoclasts, and is therefore believed to be involved in bone resorption. (Zhang et al. 2002, Bone 31(3): 359-365).

WO 98/35056 recites a method of treating or preventing osteoporosis or inflammation in a mammal by administering a compound identified by contacting the compound and PYK2 and determining if binding has occurred.

The PYK2 protein is described in, for example, U.S. Pat. No. 5,837,524.

The PYK2 protein is also described in, for example, U.S. Pat. No. 5,837,815.

Although there is a variety of therapies for individuals with disorders of bone metabolism, there is a continuing search to fill a need for alternative bone therapies. More particularly, there is a need for therapeutic agents and methods to stimulate osteoblast function increase bone formation thus restore bone mass and rebuilt bone structures in a condition with low bone mass such as osteoporosis.

### SUMMARY OF THE INVENTION

There is now provided in the present invention a method of stimulating osteoblast function comprising administering a PYK2 inhibitor to a mammal in need thereof in an amount effective to stimulate an osteoblast function.

Desirably, a PYK2 inhibitor useful in the present invention inhibits PYK2-dependant kinase activity.

Optionally, a PYK2 inhibitor useful in the present invention is a direct PYK2 inhibitor.

The present invention is useful to treat a mammal that can benefit from stimulating osteoblast function. A mammal that can benefit from stimulating osteoblast function is a mammal that is in need of augmenting and maintaining bone mass, preventing bone loss, and/or stimulating osteoblast function in a local region of the skeleton.

Osteoblast function, according to the present invention, includes without limitation, bone formation, metabolic activity that contributes towards bone formation, and metabolic activity that is associated with osteoblast phenotype. Such function can be as demonstrated in vivo, in vitro, or ex vivo.

Optionally, the present invention further comprises administration of a second therapeutic bone agent.

Optionally, the second therapeutic bone agent is an anti-resorptive agent and/or an anabolic bone agent.

Another aspect of the present invention is a method to identify a PYK2 inhibitor effective as a therapeutic bone agent comprising administering a test agent to an osteoblast-like cell and determining if osteoblast function is stimulated.

Optionally, the identifying method further comprises contacting the test agent with PYK2 and determining if PYK2 activity is inhibited.

PYK2 activity can be assessed by determining PYK2 dependant phosphorylation of endogenous substrates including PYK2 and by phosphorylation of exogenously added substrates, wherein said substrates can be natural or artificial.

### BRIEF DESCRIPTION OF THE DRAWING(S)

FIG. 1 is an SDS-PAGE blot illustrating PYK2 expression in murine and human osteoblasts as described in Example 1 herein.

FIG. 2 is a graph illustrating greater alkaline phosphatase activity resulting from culturing murine MSC with a PYK2 inhibitor as described in Example 2 herein.

FIG. 3 is a graph illustrating greater calcium deposition in vitro in murine MSC after being cultured with a PYK2 inhibitor as described in Example 2 herein.

FIG. 4 is a graph showing greater alkaline phosphatase activity of human MSC after being cultured with a PYK2 inhibitor as described in Example 3 herein.

FIG. 5 is a graph illustrating greater calcium deposition of human MSC treated with a PYK2 inhibitor as compared to control MSC as described in Example 3 herein.

FIG. 6 is a graph illustrating increased alkaline phosphatase activity in murine MC3T3 cells cultured with a PYK2 inhibitor as described in Example 4 herein.

FIG. 7 is a SDS-PAGE blot Illustrating the inhibition of stimulated phosphorylation of tyrosine 402 of PYK2 in MC3T3 cells by the PYK2 inhibitor, PF—Y as described in Example 5 herein.

FIG. 8 is a graph illustrating the faster differentiation (as viewed by alkaline phosphatase activity) of PYK2 knock out mesenchymal stem cells (MSC) as compared to control MSC as described in Example 6 herein.

FIG. 9 is a graph illustrating greater calcium deposition of PYK2 KO osteoblasts in vitro as compared to control osteoblasts as described in Example 6 herein.

FIG. 10 is a photographic representation illustrating greater mineralization of differentiated PYK2 KO osteoblasts as compared to control osteoblasts after 21 days in culture as described in Example 6 herein.

FIG. 11 is a photographic representation of micro-computerized tomography analysis of distal femoral metaphysis showing a significant increase in bone mass in PYK2 knockout mice compared with wild-type controls at 6 months of age as described in Example 7 herein.

FIG. 12 is photographic representations illustrating a higher bone mass (micro-CT images, right panel) and greater bone formation (histomorphometric images, left panel) in lumber vertebral body of 6 month-old PYK2 knockout female mice as compared with wild-type littermate controls (C57BI/6) as described in Example 7 herein.

### DETAILED DESCRIPTION OF THE INVENTION

As used herein, the following definitions apply:

“PYK2 inhibition” means inhibition of PYK2 function.

“PYK2-dependant phosphorylation” means the phosphorylation activity of PYK2 irrespective of the substrate phosphorylated. PYK2-dependant phosphorylation is to be distinguished from the term “PYK2 phosphorylation” which denotes the phosphorylation of PYK2, which includes auto-phosphorylation (self, e.g. known to occur at Y402) or trans-phosphorylation (by, for example, Src, known to occur at Y-579, 580).

A “selective PYK2 inhibitor” means a PYK2 inhibitor that has a greater in vitro IC50 towards PYK2 than towards c-erbB-2, c-met, tle-2, PDGFr, FGFr, c-Src, or VEGFR.

A “direct PYK2 inhibitor” means a PYK2 inhibitor wherein inhibition results, in part, from a direct physical interaction between the inhibitor and PYK2.

A “PYK2 inhibitor” includes pharmaceutically acceptable salts.

“Pharmaceutically acceptable” means that the carrier, diluent, excipients, salt, solvate, and/or hydrate must be compatible with the other Ingredients of the formulation, and not deleterious to the recipient thereof.

“PYK2 inhibitor” includes a prodrug made therefrom.

“Prodrug” refers to compounds that are drug precursors which following administration, release the drug in vivo via some chemical or physiological process (e.g., a prodrug on being brought to the physiological pH or through enzyme action is converted to the desired drug form). Prodrugs for compounds of Formula I are disclosed in U.S. Patent Application Ser. No. 60/435,670, hereby incorporated by reference.

“Therapeutic agent” means an agent that is useful to treat a mammal.

“Treat”, “treating”, or “treatment” includes preventative (e.g., prophylactic) and palliative treatment, as well a corrective treatment.

“Osteoblast-like cells” means cells that express, or can be manipulated in culture in such a way that causes to be expressed, an osteoblast function.

A “PYK2 pseudosubstrate” is a substrate that comprises the PYK2 tyrosine 402 phosphorylation site SESCSIESDIYAEIPDETLR, but is lacking at least one other PYK2 region such as the ezrin/radixin/moesin protein domain, the focal adhesion targeting region, or any other region of at least 100 amino acid residues.

“Pharmaceutically acceptable salt(s)” Includes salts of acidic or basic groups that may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

One embodiment of the present invention is a method of stimulating osteoblast function comprising administering an amount of a PYK2 inhibitor to a mammal in need thereof wherein said amount is effective to stimulate an osteoblast function.

In one embodiment, the PYK2 inhibitor is a selective inhibitor. Optionally, a selective PYK2 inhibitor embraces a PYK2 inhibitor that has inhibitor activity towards FAK.

In another embodiment, the PYK2 inhibitor is a direct inhibitor. Optionally, the direct inhibitor exhibits a direct physical interaction that is noncovalent. Optionally, a direct inhibitor has an equilibrium binding constant (i.e. Ka) for PYK2 of at least about 1000 nM. Optionally, the Ka is at least about 300 nM. One skilled in the art is readily able to determine Ka using any number of physicobiochemical methods, for example, a BioCore 3000 (BioCore Medical Technologies, Inc.).

A mammal in need of treatment according to the present invention includes a mammal wherein it is desirable to stimulate an osteoblast function. Such a mammal includes humans, companion animals (e.g. dogs, cats, other domesticated mammals, etc.) and agriculturally-relevant mammals (e.g. cows, pigs, sheep, horses, etc.).

According to the present invention, conditions wherein it is desirable to stimulate an osteoblast function include, by non-limiting example, a condition selected from osteoporosis, osteopenia, bone fracture, osteomalacia, rickets, fibrogenesis imperfecta ossium, periodontitis, low bone density, and conditions at risk thereof

Further conditions wherein it is desirable to stimulate an osteoblast function include condition(s) which presents with low bone mass. The phrase “condition(s) which presents with low bone mass” refers to a condition where the level of bone mass is below the age specific normal. For example, age specific normal is defined in standards by the World Health Organization “Assessment of Fracture Risk and its Application to Screening for Postmenopausal Osteoporosis (1994). Report of a World Health Organization Study Group. World Health Organization Technical Series 843” (pages 1-29). Included in “condition(s) which presents with low bone mass” are primary and secondary osteoporosis. Secondary osteoporosis includes glucocorticoid-induced osteoporosis, hyperthyroidism-induced osteoporosis, immobilization-induced osteoporosis, heparin-induced osteoporosis and immunosuppressive-induced osteoporosis. Also included is periodontal disease, alveolar bone loss, osteotomy and childhood idiopathic bone loss.

Optionally, osteoporosis conditions can be of a type selected from glucocorticoid-induced osteoporosis, hyperthyroidism-induced osteoporosis, immobilization-induced osteoporosis, heparin-induced osteoporosis, post-menopausal osteoporosis, and vitamin D deficient and Immunosuppressive-induced osteoporosis.

The “condition(s) which presents with low bone mass” also includes long term complications of osteoporosis such as curvature of the spine, loss of height and prosthetic surgery.

The phrase “condition which presents with low bone mass” also refers to a condition known to result in a significantly higher than average risk of developing such diseases as are described herein including osteoporosis (e.g., post-menopausal women, men over the age of 60, Individuals who smoke, individuals who consume higher than average amounts of alcohol, have a sedentary lifestyle, low calcium intake, have low body weight, individuals with a family history of low bone mass or hip fracture, etc.).

Further conditions wherein it is desirable to stimulate an osteoblast function further include a condition where bone loss occurs with time at a rate greater than that of the age- and gender-matched population. By non-limiting example, such a condition can be selected from conditions including osteoporosis, osteoarthritis, rheumatoid arthritis, bone loss associated with periodontitis, alveolar bone loss, and childhood idiopathic bone loss.

Further conditions wherein it is desirable to stimulate an osteoblast function further include, by non-limiting example, a surgical procedure. Exemplary procedures include facial reconstruction, maxillary reconstruction, mandibular reconstruction, bone graft, prosthetic implant, and vertebral synostosis

Further conditions wherein it is desirable to stimulate an osteoblast function a condition wherein it is desirable to enhance long bone extension.

Further conditions wherein it is desirable to stimulate an osteoblast function are conditions wherein the subject is at risk of one of the above-mentioned conditions.

A useful dosage is about 0.001 to about 100 mg/kg/day of PYK2 inhibitor. An optional dosage is about 0.01 to about 10 mg/kg/day of PYK2 inhibitor.

### PYK2 Inhibitors

A PYK2 inhibitor, as used in the present invention can be any agent that inhibits PYK2 function, for example, a small molecule inhibitor. Desirably, a small molecule Inhibitor has a molecular weight of less than 2000 Daltons.

Methods for the identification of a PYK2 inhibitor according to the present invention are given in, for example, U.S. Pat. No. 5,837,524 hereby incorporated by reference.

Other methods for the identification of a PYK2 inhibitor according to the present invention are given in, for example, U.S. Pat. No. 5,837,815, hereby incorporated by reference. These methods may include, for example, assays to Identify agents capable of disrupting or inhibiting or promoting the interaction between components of the complexes, such as between PYK2 and NBP, gelosin, Src kinase, paxillin, CAS120 and the like.

Other methods for the identification of a PYK2 inhibitor are given in the examples herein.

Additionally, a PYK2 inhibitor can be identified by its ability to Inhibit PYK2 activity as set forth bellow (“PYK2 Inhibition”).

FAK protein tyrosine kinase inhibitors belonging to the genus of Formula I (described below) are also PYK2 inhibitors and are useful in the present invention. The compounds of Formula I are described in co-assigned U.S. application 60/435,670 (filed 20 Dec. 2002) hereby incorporated by reference.

The compounds of Formula I are also described in co-assigned U.S. application 60/500,742 (filed 5 Sep. 2003), hereby incorporated by reference.

The compounds of Formula I are also described in co-assigned U.S. application Ser. No. 10/734,039 (filed 11 Dec. 2003), hereby incorporated by reference.

The compounds of Formula I are also described in co-assigned U.S. application Ser. No. 10/733,215 (filed 11 Dec. 2003), hereby incorporated by reference.

The compounds of Formula I are also described in co-assigned U.S. application 60/571,312 (filed 14 May 2004), hereby incorporated by reference.

The compounds of Formula I are also described in co-assigned U.S. application 60/571,210 (filed 14 May 2004), hereby incorporated by reference.

The compounds of Formula I are also described in co-assigned U.S. application 60/571,209 (filed 14 May 2004), hereby Incorporated by reference.

Formula I compounds comprise a broad class of trifluoromethylpyrimidine compounds represented below with the proviso that the “A” and “Ar substitutions are those provided for by U.S. Patent Application Ser. No. 60/435,670, hereby incorporated by reference.

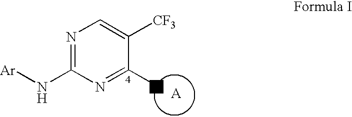

Optionally, Formula I compounds useful according to the present invention comprise 5-aminooxindole compounds as described in U.S. patent application Ser. No. 10/733,215, hereby incorporated by reference. Such compounds are shown generically as Formula II below, with the proviso that “A” substitutions are those provided for by U.S. patent application Ser. No. 10/733,215, hereby incorporated by reference.

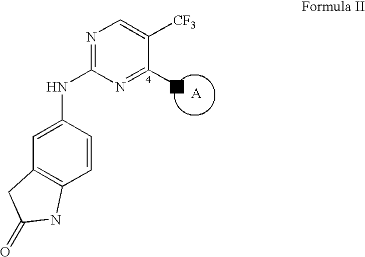

Optionally, Formula I compounds useful according to the present invention comprise tertiary aminopyrimidine compounds as described in U.S. patent application Ser. No. 10/734,039, filed 12 Dec. 2003; hereby incorporated by reference. Such compounds are shown generically herein as Formula III below, with the proviso that the substituents “Ar, R1, R2, R3, R4, and n” are those substituents set forth in U.S. patent application Ser. No. 10/734,039, hereby incorporated by reference.

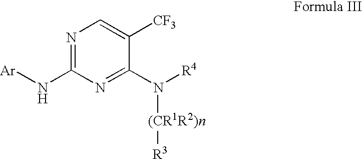

By way of example, a PYK2 inhibitor useful according to the present invention, is a compound PF—X illustrated below, a species of Formula I, Formula II, and Formula III.

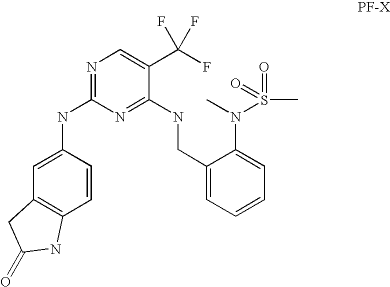

PF—X structure and generic synthesis is disclosed in U.S. Patent Application Ser. No. 60/435,670, filed Dec. 20, 2002, hereby incorporated by reference.

PF—X structure and generic synthesis is also disclosed in U.S. patent application Ser. No. 10/734,039, filed 11 Dec. 2003; hereby incorporated by reference.

Optionally, a PYK2 inhibitor useful according to the present invention is selected from compounds that block the signally pathway of Flk-1 receptor, for example compound PF—Y Illustrated below.

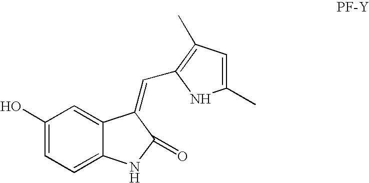

PF—Y structure and synthesis is disclosed in U.S. patent application Ser. No. 09/569,545 (Publication Number US 2003/0191162 A1) filed 12 May 2000; hereby incorporated by reference.

Combination Treatment.

The present invention can optionally further comprise administration of a second therapeutic bone agent. Such useful bone therapeutic agents can be any anti-resorptive agent or bone anabolic agent or an agent that is anti-resorptive and bone anabolic.

The use of the term “second therapeutic bone agent” herein, embraces more than one bone agent. As described herein, the term “second therapeutic bone agent” does not imply any order of administration (relative to a PYK2 inhibitor) and can be administered before, after, or simultaneously with a PYK2 inhibitor

Any antiresorptive agent can optionally be used as the second therapeutic bone agent in this invention, including without limitation, an estrogenic compound, a selective estrogen receptor modulator, or a bisphosphonate.

By way of example only, it has been reported (Osteoporosis Conference Scrip No. 1812/13 Apr. 16/20, 1993, p. 29) that raloxifene, 6-hydroxy-2(4-hydroxyphenyl)-3-[4-(2-piperidinoethoxy)benzoyl]benzo[b]thiophene.

Raloxifene mimics the favorable action of estrogens on bone and lipids but, unlike estrogen, has minimal uterine stimulatory effect. [Black, L. J. et al., Raloxifene (LY139481HCl) Prevents Bone Loss and Reduces Serum Cholesterol Without Causing Uterine Hypertrophy in Ovariectomized Rats.

A related study showing such selective effects was reported in J. Clin. Invest., 1994, 93, 6369 and Delmas, P. D. et al.

Yet another study showed selective effects of raloxifene and was reported in New England Journal of Medicine, 1997, 337, 1641-1647].

Also, tamoxifen, 1-(4-b-dimethylaminoethoxyphenyl)-1,2-diphenyl-but-1-ene, is an anti-estrogen that is proposed as an osteoporosis agent which has a palliative effect on breast cancer, but is reported to have some estrogenic activity in the uterus.

U.S. Pat. No. 5,254,595 discloses agents such as droloxifene, which prevent bone loss, reduce the risk of fracture and are useful for the treatment of osteoporosis, hereby incorporated by reference.

U.S. Pat. No. 5,552,412, hereby incorporated by reference, discloses selective estrogen receptor modulator (SERM) compounds of the formula

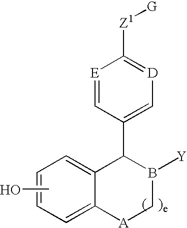

wherein the variables are defined as set forth therein. Cis-6-phenyl-5-(4-2-pyrrolidin-1-yl-ethoxy)-phenyl)-5,6,7,8,-tetrahydronaphthalene-2-ol, and more particularly (−)-Cis-6-phenyl-5-(4-(2-pyrrolidin-1-yl-ethoxy)-phenyl)-5,6,7,8,-tetahydronaphthalene-2-ol is an orally active, highly potent SERM which prevents bone loss, decreases total serum cholesterol, and does not have estrogen-like uterine stimulating effects in OVX rats. U.S. Pat. No. 5,948,809, also incorporated herein by reference, discloses (−)-Cis-6-phenyl-4-(2-pyrrolidin-1-yl-ethoxy)-phenyl)-5,6,7,8,-tetrahydronaphthalene-2-ol, tartrate salt.

Any bone anabolic agent can optionally be used as a second therapeutic bone agent of this invention. Including without limitation IGF-1 optionally with IGF-1 binding protein 3, IGF-II, prostaglandin, prostaglandin agonist/antagonist, sodium fluoride, parathyroid hormone (PTH), active fragments of parathyroid hormone, parathyroid hormone related peptides and active fragments and analogues of parathyroid hormone related peptides, growth hormone or growth hormone secretagogues and the pharmaceutically acceptable salts thereof.

Optionally, a second therapeutic bone agent, useful according to the present invention, is a prostaglandin agonist. Optionally, the prostaglandin agonist is a PGE2 EP2 selective receptor agonist. Non-limiting examples of EP₂ selective receptor agonists are agonists of Formula AA as set forth in U.S. Pat. No. 6,498,172, hereby incorporated by reference.

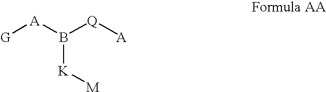

Other EP₂ selective receptor agonists that can be used in the present invention include the prostaglandin receptor agonists disclosed in U.S. Pat. No. 6,288,120, hereby incorporated by reference.

Other EP₂ selective receptor agonists that can be used in the present invention include the prostaglandin receptor agonists disclosed in U.S. Pat. No. 6,124,314, hereby incorporated by reference.

An optional EP2 selective receptor agonist is 7-[(4-butyl-benzyl)-methanesulfonyl-amino]-heptanoic acid or a pharmaceutically acceptable salt or prodrug thereof, or a salt of a prodrug disclosed in U.S. Pat. No. 6,288,120, hereby incorporated by reference. An optional salt of 7-[(4-butyl-benzyl)-methanesulfonyl-amino]-heptanoic acid is the monosodium salt.

Optionally, an EP2 receptor agonist is (3-(((4-tert-butyl-benzyl)-(pyridine-3-sulfonyl)-amino)-methyl)-phenoxy)-acetic acid, or a pharmaceutically acceptable salt or prodrug thereof, or a salt of a prodrug. An option salt is the sodium salt. The (3-(((4-tert-butyl-benzyl)-pyridine-3-sulfonyl)-amino)-methyl)phenoxy)-acetic acid compounds are set forth in U.S. Pat. No. 6,498,172, hereby incorporated by reference.

### Dosing

The amount (and timing) of PYK2 inhibitors and/or a second therapeutic bone agent administered will, of course, be dependent on the subject being treated, on the severity of the affliction, on the manner of administration and on the judgment of the prescribing physician. Thus, because of patient to patient variability, the dosages given below are a guideline and the physician may titrate doses of the drug to achieve the treatment (e.g., bone mass augmentation) that the physician considers appropriate for the patient. In considering the degree of treatment desired, the physician must balance a variety of factors such as bone mass starting level, age of the patient, presence of preexisting disease, as well as presence of other diseases (e.g., cardiovascular disease).

Optionally, an amount of a PYK2 inhibitor and/or a second therapeutic bone agent of this invention are used that is sufficient to augment bone mass to a level that is above the bone fracture threshold (as detailed in the World Health Organization Study previously cited herein).

The amount of a bone anabolic agent to be used is determined by, for example, “In Vivo Assay of Bone Formation” as set forth below.

In general an effective dosage for an anabolic agent is in the range of 0.001 to 100 mg/kg/day, preferably 0.01 to 50 mg/kg/day.

The amount of the anti-resorptive agent to be used is determined by its activity as a bone loss inhibiting agent. A therapeutic dose can be further determined by means of an individual agent's pharmacokinetics and its minimal maximal effective dose in inhibition of bone loss using a protocol such as described herein (Assay For Determining Activity For Preventing Estrogen Deficiency-Induced Bone Loss).

In general, an effective dosage for an anti-resorptive agent is about 0.001 mg/kg/day to about 20 mg/kg/day.

### Co-administration Regimen.

In one embodiment of the present invention, a PYK2 inhibitor and a second therapeutic bone agent are co-administered simultaneously or sequentially in any order, or a single pharmaceutical composition comprising a PYK2 inhibitor as described above and a second therapeutic agent as described above in a pharmaceutically acceptable carrier can be administered. The second therapeutic bone agent can be a bone anabolic agent, an anti-resorptive agent, and/or an agent that is anti-resorptive and bone anabolic.

For example, a PYK2 antagonist can be used alone or in combination with a second therapeutic bone agent for about one week to about three years, followed by a second therapeutic bone agent alone for about one week to about thirty years, with optional repeat of the full treatment cycle.

Alternatively, for example, a PYK2 antagonist can be used alone or in combination with a second therapeutic bone agent for about one week to about thirty years, followed by a second therapeutic bone agent alone for the remainder of the patient's life.

Alternatively, for example, a PYK2 inhibitor as described above may be administered once daily and a second therapeutic bone agent as described above (e.g., estrogen agonist/antagonist) may be administered daily in single or multiple doses.

Alternatively, for example, the PYK2 inhibitor and a bone therapeutic agent may be administered sequentially wherein the PYK2 inhibitor as described above may be administered once daily for a period of time sufficient to augment bone mass to a level which is above the bone fracture threshold. Optionally, the fracture threshold is as set forth by the World Health Organization Study “Assessment of Fracture Risk and its Application to Screening for Postmenopausal Osteoporosis (1994). Report of a World Health Organization Study Group. World Health Organization Technical Series 843, pages 1-29). Following the PYK2 inhibitor administration, a second therapeutic bone agent can be administered, daily in single or multiple doses. Optionally, the PYK2 inhibitor as described above is administered once daily in a rapid delivery form such as oral delivery (e.g., sustained release delivery form is preferably avoided).

In an optional aspect of the present invention, a PYK2 inhibitor and a second therapeutic bone agent are administered substantially simultaneously.

In an optional aspect of the present invention, a PYK2 inhibitor is administered for a period of from about one week to about thirty years.

Optionally the administration of a PYK2 inhibitor is followed by administration of a second therapeutic bone agent wherein the second therapeutic bone agent is a selective estrogen receptor modulator administered for a period of from about three months to about thirty years without the administration of the first agent during the second period of from about three months to about thirty years.

Alternatively, the administration of a PYK2 inhibitor is followed by administration of a second therapeutic bone agent wherein the second therapeutic bone agent is a selective estrogen receptor modulator administered for a period greater than about thirty years without the administration of the first agent during the greater than about thirty year period.

### Route of Administration.

Administration of the agents of this invention can be via any method that delivers an agent of this invention systemically and/or locally (e.g., at the site of the bone fracture, osteotomy, or orthopedic surgery). These methods include oral routes, parenteral, intraduodenal routes, etc. Generally, the agents of this invention are administered orally, but parenteral administration (e.g., intravenous, intramuscular, subcutaneous or intramedullary) may be utilized, for example, where oral administration is inappropriate for the target or where the patient is unable to ingest the drug.

The PYK2 inhibitors and optional second therapeutic bone agent can be used for the treatment and promotion of healing of bone fractures and osteotomies by the local application (e.g., to the sites of bone fractures or osteotomies) of the agents of this invention or compositions thereof. The agents of this invention are applied to the sites of bone fractures or osteotomies, for example, either by injection of the agent in a suitable solvent (e.g., an oily solvent such as arachis oil) to the cartilage growth plate or, in cases of open surgery, by local application thereto of such agents in a suitable carrier such as bone-wax, demineralized bone powder, polymeric bone cements, bone sealants etc. Alternatively, local application can be achieved by applying a solution or dispersion of the agent in a suitable carrier onto the surface of, or incorporating it into solid or semi-solid implants conventionally used in orthopedic surgery, such as dacron-mesh, Gore-tex®, gel-foam and kiel bone, or prostheses.

A PYK2 inhibitor and an optional second therapeutic bone agent of this invention can be administered systemically and/or applied locally to the site of a fracture or osteotomy in a suitable carrier in combination with one or more bone therapeutic agents described above.

In the present invention, a PYK2 inhibitor and an optional second therapeutic bone agent are generally administered in the form of a pharmaceutical composition comprising at least one of the agents of this invention together with a pharmaceutically acceptable vehicle or diluent. Thus, the agents of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinopyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the agents of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the agent(s) of this invention, preferably 1%-70%. In any event, the composition or formulation to be administered will contain a quantity of an agent(s) according to the invention in an amount effective to treat the disease/condition of the subject being treated, e.g., a bone disorder.

### Methods of Identifying a Therapeutic Agent that Stimulates Osteoblast Function

One aspect of the present invention is a method to identify a PYK2 inhibitor effective as a therapeutic bone agent comprising administering a test agent to an osteoblast-like cell and determining if osteoblast function is stimulated. Optionally, the identifying method further comprises contacting the test agent with PYK2 and determining if PYK2 activity is inhibited.

The effect of a test agent on PYK2 activity can be determined either in vivo or in vitro according to any method known to one skilled in the art, for example, any of the methods taught herein.

In one embodiment, the effect of a test agent on PYK2 activity is determined in vitro in a whole cell or a cell-free assay. For the whole cell assay, the cells can be intact or disrupted. The cells can be osteoblast-like cells or an osteoblast surrogate cell model.

The effect of the test agent on osteoblast function can be determined ex vivo, in vivo or in vitro according to any method known to one skilled in the art, for example, any of the methods taught herein.

In one embodiment, the effect of a test agent on PYK2 activity and the effect of the test agent on osteoblast function are determined in vitro. Optionally, the in vitro determination of PYK2 activity is conducted in cultured osteoblast-like cells or a suitable osteoblast surrogate model expressing endogenous or recombinant PYK2, or in a cell-free in vitro assay.

In another embodiment, the effect of a test agent on PYK2 activity is determined in vitro and the effect of the test agent on osteoblast function is determined in vivo.

In another embodiment, the effect of a test agent on PYK2 activity is determined in vivo and the effect of the test agent on osteoblast function is determined in vitro.

In another embodiment, the effect of a test agent on PYK2 activity and the effect of the test agent on osteoblast function are determined in vivo.

Optionally, the determination of the test agent's effect on PYK2 activity follows activating PYK2 (i.e., determining of the test agents effect on a previously activated PYK2). As a non-limiting example, PYK2 can be previously activated by Src-mediated phosphorylation as set forth below.

### Osteoblast Function.

Osteoblast function, according to the present invention, includes without limitation, one or more of bone formation, metabolic activity that contributes towards bone formation, and metabolic activity that is associated with osteoblast phenotype (“osteoblast function”). Such function can be as demonstrated in vivo, in vitro, or ex vivo.

Osteoblast function can be quantified by any means to determine one or more features generally attributed to osteoblasts in vivo. While one skilled in the art will readily understand the meaning of “features generally attributed to osteoblasts in vivo” an exemplary, non-limiting list include production of alkaline phosphatase (of the tissue non-specific type), osteopontin, PYK2, type I collagen, IGF-1, IGF-II, IGF binding proteins, extracellular matrix, insoluble extracellular minerals comprising calcium and phosphate, and mineralized matrix. When osteoblast function is determined in vivo, in addition to the previous examples, bone mass, bone strength, bone repair, histomorphometric features, and serum biomarkers can be determined. Serum biomarkers of osteoblast function can be, by way of non-limiting example, osteocalcin, bone specific alkaline phosphatase, amino-terminal propeptide of type I procollagen (P1NP) or procollagen extension peptide (P1CP).

### Osteoblast-Like Cells.

Cells recognized by the skilled artisan as osteoblast-like include MC3T3s, SAOS, ROS (e.g. ROS 17/2.8), UMR, and mesenchymal stem cells isolated from bone marrow (e.g. human, mouse).

Any osteoblast-like cells that express PYK2 (either naturally or recombinantly) may be used in accordance with the screening method of present invention. Accordingly, osteoblast-like cells is also meant to embrace cells as described above and which are transformed with a vector containing recombinant PYK2 and are capable of transcribing and translating such nucleic acids encoding PYK2. Thus, osteoblast-like cells can be cells that express endogenous PYK2, recombinant PYK2, or both. The sequence of PYK2 from several species is known, including mouse, rat, and human and one skilled in the art can readily perform transformation of osteoblast-like cells with various PYK2 constructs.

### In Vivo Assay of Bone Formation.

The usefulness and dosing of a PYK2 inhibitor or a second therapeutic bone agent of the present invention in stimulating osteoblast function can be assessed, by non-limiting example, by its ability to augment bone formation and increase bone mass. Such abilities can be tested in intact male or female rats, sex hormone deficient male (orchiectomy) or female (ovariectomy) rats.

Male or female rats at different ages (such as 3 months of age) can be used in the study. The rats are either intact or castrated (ovariectomized or orchiectomized), and subcutaneously Injected or gavaged with prostaglandin agonists at different doses (such as 1, 3, or 10 mg/kg/day) for 30 days. In the castrated rats, treatment is started at the next day after surgery (for the purpose of preventing bone loss) or at the time bone loss has already occurred (for the purpose of restoring bone mass). During the study, all rats are allowed free access to water and a pelleted commercial diet (Teklad Rodent Diet #8064, Harlan Teklad, Madison, Wis.) containing 1.46% calcium, 0.99% phosphorus and 4.96 IU/g of Vit.D 3. All rats are given subcutaneous injections of 10 mg/kg calcein on days 12 and 2 before sacrifice. The rats are sacrificed. The following endpoints are determined:

### Femoral Bone Mineral Measurements:

The right femur from each rat is removed at autopsy and scanned using dual energy x-ray absorptiometry (DXA, QDR 1000/W, Hologic Inc., Waltham, Mass.) equipped with “Regional High Resolution Scan” software (Hologic Inc., Waltham, Mass.). The scan field size is 5.08×1.902 cm, resolution is 0.0254×0.0127 cm and scan speed is 7.25 mm/second. The femoral scan images are analyzed and bone area, bone mineral content (BMC), and bone mineral density (BMD) of whole femora (WF), distal femoral metaphyses (DFM), femoral shaft (FS), and proximal femora (PF) are determined.

### Lumbar Vertebral Bone Mineral Measurements:

Dual energy x-ray absorptiometry (QbR 1000/W, Hologic, Inc., Waltham, Mass.) equipped with a “Regional High Resolution Scan” software (Hologic, Inc., Waltham, Mass.) is used to determined the bone area, bone mineral content (BMC), and bone mineral density (BMD) of whole lumbar spine and each of the six lumbar vertebrae (LV1-6) in the anesthetized rats. The rats are anesthetized by injection (i.p.) of 1 ml/kg of a mixture of ketamine/rompun (ratio of 4 to 3), and then placed on the rat platform. The scan field sized is 6×1.9 cm, resolution is 0.0254×0.0127 cm, and scan speed is 7.25 mm/sec. The whole lumbar spine scan image is obtained and analyzed. Bone area 30 (BA), and bone mineral content (BMC) is determined, and bone mineral density is calculated (MBC divided by BA) for the whole lumbar spine and each of the six lumbar vertebrae (LV1-6).

### Tibial Bone Histomorphometric Analyses:

The right tibia is removed at autopsy, dissected free of muscle, and cut into three parts. The proximal tibia and the tibial shaft are fixed in 70% ethanol, dehydrated in graded concentrations of ethanol, defatted in acetone, then embedded In methyl methacrylate (Eastman Organic Chemicals, Rochester, N.Y.).

Frontal sections of proximal tibial metaphyses at 4 and 10 μm thickness are cut using Reichert-Jung Polycut S microtome. The 4 μm sections are stained with modified Masson's Trichrome stain while the 10 μm sections remained unstained. One 4 μm and one 10 μm sections from each rat are used for cancellous bone histomorphometry.

Cross sections of tibial shaft at 10 μm thickness are cut using Reichert-Jung Polycut S microtome. These sections are using for cortical bone histomorphometric analysis.

Cancellous bone histomorphometry: A Bloquant OS/2 histomorphometry system (R&M biometrics, Inc., Nashville, Term.) is used for the static and dynamic histomorphometric measurements of the secondary spongiosa of the proximal tibial metaphyses between 1.2 and 3.6 mm distal to the growth plate-epiphyseal junction. The first 1.2 mm of the tibial metaphyseal region needs to be omitted in order to restrict measurements to the secondary spongiosa. The 4 μm sections are used to determine indices related to bone volume, bone structure, and bone resorption, while the μm sections are used to determine indices related to bone formation and bone turnover.

### Measurements and Calculations Related to Trabecular Bone Volume and Structure:

(1) Total metaphyseal area (TV, mm 2): metaphyseal area between 1.2 and 3.6 mm distal to the growth plate-epiphyseal Junction.
(2) Trabecular bone area (BV, mm 2 total area of trabeculae within TV.
(3) Trabecular bone perimeter (BS, m): the length of total perimeter of trabeculae.
(4) Trabecular bone volume (BV/TV, %): BV/TV×100.
(5) Trabecular bone number (TBN, #/mm): 1.199/2×BS/TV.
(6) Trabecular bone thickness (TBT, μm): (2000×1.199)×(BV BS).
(7) Trabecular bone separation (TBS, μm): (2000×1.199)×(TV-BV).

### Measurements and Calculations Related to Bone Resorption:

(1) Osteoclast number (OCN, #): total number of osteoclast within total metaphyseal area.
(2) Osteoclast perimeter (OCP, m): length of trabecular perimeter covered by osteoclast.
(3) Osteoclast number/mm (OCN/mm, #/mm): OCN/BS.
(4) Percent osteoclast perimeter (% OCP, %): OCP/BS×100.

### Measurements and Calculations Related to Bone Formation and Turnover:

(1) Single-calcein labeled perimeter (SLS, m): total length of trabecular perimeter labeled with one calcein label.
(2) Double-calcein labeled perimeter (DLS, m): total length of trabecular perimeter labeled with two calcein labels.
(3) Inter-labeled width (ILW, μm): average distance between two calcein labels.
(4) Percent mineralizing perimeter (PMS, %): (SLS/2+DLS)/BS×100.
(5) Mineral apposition rate (MAR, μ, m/day): IL/label interval.
(6) Bone formation rate/surface ref. (BFR/BS, μm²/d/μm): (SLS/2+DLS)×MAR/BS.
(7) Bone turnover rate (BTR, %/y): (SLS/2+DLS)×MAR/BV×100.

### Cortical Bone Histomorphometry:

Any histomorphometric analysis can be used. By way of example, a Bioquant OS/2 histomorphometry system (R&M biometrics, Inc., Nashville, Tenn.) can be used for the static and dynamic histomorphometric measurements of tibial shaft cortical bone. Total tissue area, marrow cavity area, periosteal perimeter, endocortical perimeter, single labeled perimeter, double labeled perimeter, and interlabeled width on both periosteal and endocortical surface are measured, and cortical bone area (total tissue area-marrow cavity area), percent cortical bone area (cortical area/total tissue area×100), percent marrow area (marrow cavity area/total tissue area×100), periosteal and endocortical percent labeled perimeter [(single labeled perimeter/2+double labeled perimeter)/total perimeter×100], mineral apposition rate (interlabeled width/intervals), and bone formation rate [mineral apposition ratex[(single labeled perimeter/2+double labeled perimeter)/total perimeter] are calculated.

Statistics can be calculated using StatView 4.0 packages (Abacus Concepts, Inc., Berkeley, Calif.). The analysis of variance (ANOVA) test followed by Fisher's PLSD are used to compare the differences between groups.

### Fracture Healing Assays for Effects on Fracture Healing after Systemic Administration

The usefulness and dosing of a systemically administered PYK2 inhibitor and/or a second therapeutic bone agent of the present invention for stimulating osteoblast function can be assessed by its ability to aid in fracture healing and can be evaluated by any method known to one skilled in the art.

One such fracture healing assay is illustrate below (Fracture Healing Assays For Effects On Fracture Healing After Local Administration). Another optional assay for determining efficacy of treatment with a systemically administered PYK2 inhibitor is as follows:

Fracture Technique Sprage-Dawley rats at 3 months of age are anesthetized with Ketamine. A 1 cm incision is made on the anteromedial aspect of the proximal part of the right tibia or femur. The following describes the tibial surgical technique. The incision is carried through to the bone, and a 1 mm hole is drilled 4 mm proximal to the distal aspect of the tibial tuberosity 2 mm medial to the anterior ridge. Intramedullary nailing is performed with a 0.8 mm stainless steel tube (maximum load 36.3 N, maximum stiffness 61.8 N/mm, tested under the same conditions as the bones). No reaming of the medullary canal is performed. A standardized closed fracture is produced 2 mm above the tibiofibular junction by three-point bending using specially designed adjustable forceps with blunt jaws. To minimize soft tissue damage, care is taken not to displace the fracture. The skin is closed with monofilament nylon sutures. The operation is performed under sterile conditions. Radiographs of all fractures are taken immediately after nailing, and animals with fractures outside the specified diaphyseal area or with displaced nails are excluded. The remaining animals are divided randomly into the following groups with 10-12 animals per each subgroup for testing the fracture healing. The first group receives daily gavage of vehicle (water: 100% Ethanol=95:5) at 1 ml/rat, while the others receive daily gavage from 0.01 to 100 mg/kg/day of the agent to be tested (1 ml/rat) for 10, 20, 40 and 80 days.

At 10, 20, 40 and 80 days, 10-12 rats from each group are anesthetized with Ketamine and autopsied by exsanguination. Both tibiofibular bones are removed by dissection and all soft tissue is stripped. Bones from 56 rats for each group are stored in 70% ethanol for histological analysis, and bones from another 5-6 rats for each group are stored in a buffered Ringer's solution (+4° C., pH 7.4) for radiographs and biomechanical testing which is performed.

Histological Analysis: The methods for histologic analysis of fractured bone have been previously published by Mosekilde and Bak (The Effects of Growth Hormone on Fracture Healing In Rats: A Histological Description. Bone, 14:19-27, 1993). Briefly, the fracture side is sawed 8 mm to each side of the fracture line, embedded undecalcified in methylmethacrylate, and cut frontals sections on a Reichert-Jung Polycut microtome in 8 μm thick. Masson-Trichrome stained mid-frontal sections (including both tibia and fibula) are used for visualization of the cellular and tissue response to fracture healing with and without treatment. Sirius red stained sections are used to demonstrate the characteristics of the callus structure and to differentiate between woven bone and lamellar bone at the fracture site. The following measurements are performed: (1) fracture gap-measured as the shortest distance between the cortical bone ends in the fracture, (2) callus length and callus diameter, (3) total bone volume area of callus, (4) bony tissue per tissue area inside the callus area, (5) fibrous tissue in the callus, (6) cartilage area in the callus.

### Biomechanical Analysis:

The usefulness and dosing of a locally administered PYK2 inhibitor and/or a second therapeutic bone agent of the present invention for stimulating osteoblast function can be assessed by its ability to positively affect bone biomechanical integrity.

Methods for biomechanical analysis have been previously published by Bak and Andreassen (The Effects of Aging on Fracture Healing in Rats. Calcif Tissue Int 45:292-297, 1989).

Other biomechanical analytical methods useful with the present invention have been previously published by Peter et al. (Peter, C. P.; Cook, W. O.; Nunamaker, D. M.; Provost, M. T.; Seedor, J. G.; Rodan, G. A. Effects of Alendronate On Fracture Healing And Bone Remodeling In Dogs. J. Orthop. Res. 14:74-70, 1996).

Briefly, radiographs of all fractures are taken prior to the biomechanical test. The mechanical properties of the healing fractures are analyzed by a destructive three- or four-point bending procedure. Maximum load, stiffness, energy at maximum load, deflection at maximum load, and maximum stress are determined.

Assay for Effects on Fracture Healing after Local Administration

The usefulness and dosing of a locally administered PYK2 inhibitor and/or a second therapeutic bone agent of the present invention for stimulating osteoblast function can be assessed by its ability to aid in fracture healing and can be evaluated by any method known to one skilled in the art.

One such fracture healing assay is set forth above (Fracture Healing Assays For Effects On Fracture Healing After Systemic Administration). Another such optional fracture healing assay useful for assessing treatment with a locally administered PYK2 inhibitor is as follows:

Fracture Technique: female or male beagle dogs at approximately 2 years of age are used in the study. Transverse radial fractures are produced by slow continuous loading in three-point bending as described by Lenehan et al. (Lenehan, T. M.; Balilgand, M.; Nunamaker, D. M.; Wood, F. E.: Effects of EHDP on Fracture Healing in Dogs. J Orthop Res 3:499-507; 1985). The wire is pulled through the fracture site to ensure complete anatomical disruption of the bone. Thereafter, local delivery of prostaglandin agonists to the fracture site is achieved by slow release of agent delivered by slow release pellets or Alzet minipumps for 10, 15, or 20 weeks.

Histological Analysis: The methods for histologic analysis of fractured bone have been previously published by Peter et al. (Peter, C. P.; Cook, W. O.; Nunamaker, D. M.; Provost, M. T.; Seedor, J. G.; Rodan, G. A Effects of alendronate on fracture healing and bone remodeling in dogs. J. Orthop. Res. 14:74-70, 1996) and Mosekilde and Bak (The Effects of Growth Hormone on Fracture Healing in Rats: A Histological Description. Bone, 14:19-27, 1993). Briefly, the fracture side is sawed 3 cm to each side of the fracture line, embedded undecalcified in methylmethacrylate, and cut on a Reichert-Jung Polycut microtome in 8 μm thick of frontal sections. Masson-Trichrome stained mid-frontal sections (including both tibia and fibula) are used for visualization of the cellular and tissue response to fracture healing with and without treatment. Sirius red stained sections are used to demonstrate the characteristics of the callus structure and to differentiate between woven bone and lamellar bone at the fracture site.

The following measurements are performed:
(1) fracture gap—measured as the shortest distance between the cortical bone ends in the fracture,
(2) callus length and callus diameter,
(3) total bone volume area of callus,
(4) bony tissue per tissue area inside the callus area,
(5) fibrous tissue in the callus,
(6) cartilage area in the callus.

Biomechanical Analysis: While the skilled artisan will recognize that a variety of methods are available for biomechanical analysis, a non-limiting example of is set forth above in “Fracture Healing Assays For Effects On Fracture Healing After Systemic Administration”.

### Assay for Determining Activity for Preventing Estrogen Deficiency-Induced Bone Loss

The usefulness and dosing of a PYK2 inhibitor and/or a second therapeutic bone agent of the present invention for stimulating osteoblast function can be assessed by its ability to prevent osteoporosis and can be evaluated by any method known to one skilled in the art.

One such method is an ovariectomized rat bone loss model of postmenopausal bone loss.

Sprague-Dawley female rats (Charles River, Wilmington, Mass.) at different ages (such as 5 months of age) are used in these studies. The rats are singly housed in 20 cm×32 cm×20 cm cages during the experimental period. All rats are allowed free access to water and a pelleted commercial diet (Agway ProLab 3000, Agway County Food, Inc., Syracuse, N.Y.) containing 0.97% calcium, 0.85% phosphorus, and 1.05 IU/g of Vlt.D 3.

A group of rats (8 to 10) are sham-operated and treated p.o. with vehicle (10% ethanol and 90% saline, 1 ml/day), while the remaining rats are bilaterally ovariectomized (OVX) and treated with either vehicle (p.o.), a PYK2 inhibitor, 17β-estradiol (Sigma, E-8876, E 2, 30 μg/kg, daily subcutaneous injection), or a selective estrogen receptor modulator (such as droloxifene at 5, 10, or 20 mg/kg, daily p.o.) for a certain period (such as 4 weeks). All rats are given subcutaneous injections of 10 mg/kg calcein (fluorochrome bone marker) 12 and 2 days before being sacrificed in order to examine the dynamic changes in bone tissue. After 4 weeks of treatment, the rats are sacrificed and autopsied. The following endpoints are determined:

Body Weight Gain: body weight at autopsy minus body weight at surgery.

Uterine Weight and Histology: The uterus is removed from each rat during autopsy, and weighed immediately. Thereafter, the uterus is processed for histologic measurements such as uterine cross-sectional tissue area, stromal thickness, and luminal epithelial thickness.

Total Serum Cholesterol: Blood is obtained by cardiac puncture and allowed to clot at 4° C., and then centrifuged at 2,000 g for 10 min. Serum samples are analyzed for total serum cholesterol using a high performance cholesterol calorimetric assay (Boehringer Mannheim Biochemicals, Indianapolis, Ind.).

Femoral Bone Mineral Measurements: While the skilled artisan will recognize that a variety of methods are available for femoral bone mineral measurements, a non-limiting example of is set forth above in “In Vivo Assay of Bone Formation”.

Proximal Tibial Metaphyseal Cancellous Bone Histomorphometric Analyses: While the skilled artisan will recognize that a variety of methods are available for histomorphometric analyses of proximal Ubial metaphyseal cancellous bone, a non-limiting example is that set forth for above in “In Vivo Assay of Bone Formation”.

### Combination Treatment Protocol

The usefulness and dosing of a PYK2 inhibitor of the present invention, in combination with a second therapeutic bone agent according to the present invention, can be evaluated by any method known to one skilled in the art including the methods described herein.

While it should readily be recognized the following protocol can be varied by those skilled in the art, an additional exemplary method is as follows:

Intact male or female rats, sex hormone deficient male (orchidectomy) or female (ovariectomy) rats may be used. In addition, male or female rats at different ages (such as 12 months of age) can be used in the studies. The rats can be either intact or castrated (ovarlectomized or orchidectomized), and administrated with a PYK2 inhibitor of the present invention at different doses for a certain period (such as two weeks to two months), and followed by administration of any anabolic agent and/or any anti-resorptive agent such as droloxifene at different doses (such as 1,5,10 mg/kg/day) for a certain period (such as two weeks to two months), or a combination treatment with both a PYK2 inhibitor and a bone therapeutic agent (e.g. and anti-resorptive agent) at different doses for a certain period (such as two weeks to two months).

In castrated rats, treatment can be started at the next day after surgery (for the purpose of preventing bone loss) or at the time bone loss has already occurred (for the purpose of restoring bone mass).

The rats are sacrificed under ketamine anesthesia. The following endpoints are determined:

I. Femoral Bone Mineral Measurements:

II. Lumbar Vertebral Bone Mineral Measurements:

III. Proximal Tibial Metaphyseal Cancellous Bone Histomorphometric Analyses:

IV. Measurements and calculations related to trabecular bone volume and structure:

V. Measurements and calculations related to bone resorption:

VI. Measurements and calculations related to bone formation and turnover:

VII. Statistics

While the skilled artisan will recognize that a variety of methods are available to determine the above-mentioned endpoints, a non-limiting example of each determination method is set forth above in “In Vivo Assay of Bone Formation”.

### PYK2 Inhibition

Inhibition of PYK2 function, according to the present invention, is determined in osteoblast-like cells (in vivo, in vitro, or ex vivo) or a suitable osteoblast surrogate. By nonlimiting example, a suitable osteoblast surrogate is an NIH3T3 gene switch cell, a PC12 neuronal cell, or primary lymphocytes.

In one embodiment, the PYK2 function inhibited is PYK2-dependant phosphorylation (i.e. tyrosine kinase activity).

Tyrosine kinase activity can be assessed by determining PYK2-dependant phosphorylation of an endogenous substrate such as PYK2 or by phosphorylation of an exogenously added substrate. An exogenously added substrate can be a natural substrate or an artificial substrate.

Optionally, phosphorylation of a substrate is measured at a tyrosine residue. Optionally, the tyrosine residue is a PYK2 tyrosine residue.

In one embodiment of the present invention, phosphorylation of PYK2 tyrosine 402 is measured. By way of a non-limiting example, PYK2 tyrosine 402 phosphorylation is determined by using an antibody that is specific for PYK2 having phosphorylated tyrosine 402. One such primary antibody suitable for the present invention is pyk2 phospho-Y402 from Biosource (catalog #44-618G).

By way of non-limiting example, PYK2-dependant phosphorylation can be measured in accordance with this invention by an in vitro kinase assay. In this assay, PYK2-dependant phosphorylation is determined by measuring the ability of PYK2 to incorporate a phosphate into a substrate. Optionally, the phosphate is labeled. Optionally, the phosphate is radiolabeled.

PYK2-dependant phosphorylation can also be measured using gamma-³²P labeled ATP as set forth, by way of example, in Example 4 of WO 98/35016, Incorporated herein by reference.

PYK2-dependant phosphorylation can also be measured in accordance with this invention by measuring the ability of PYK2 to phosphorylate PYK2 at tyrosine residue 402. This assay is generally performed using conditions similar to those for the in vitro kinase assay using poly-(glu,tyr) as described intra, except that no exogenous substrate is required to be present. In an optional embodiment, the phosphate is radiolabeled and its incorporation into PYK2 is monitored by SDS-PAGE followed by X-ray radiography. The amount of phosphorylation of PYK2 generally reflects the activation state of PYK2. Thus, a compound that Inhibits PYK2 dependent phosphorylation of PYK2 would be a PYK2 inhibitor.

In another example, PYK2-dependant phosphorylation can be measured by using antibody specific for phosphorylated PYK2, as illustrated in Example 5. The amount of antibody specific for phosphorylated PYK2 (visualized, for example, by Western Blot) can be normalized to the amount of antibody specific for PYK2 (i.e., antibody that immunoreacts with phosphorylated and non phosphorylated PYK2).

PYK2-dependant phosphorylation can also be measured in accordance with this Invention by determining labeled phosphate incorporation into an exogenously added substrate. A potential PYK2 inhibitor and an endogenous PYK2 substrate are added to PYK2, and incorporation is quantified in the presence and absence of the putative PYK2 inhibitor. In this embodiment, PYK2 can be recombinant, from a natural (mammalian source), or provided in an intact or a disrupted osteoblast-like cell.

### PYK2 Pseudosubstrate

In another embodiment, PYK2-dependant phosphorylation (or Inhibition thereof) can be quantified using an exogenous substrate comprising a PYK2 pseudosubstrate. A PYK2 pseudosubstrate can contain any N or C terminal modification such as, by non-limiting example, biotin. A cysteine residue can be modified or substituted with serine to prevent disulphide formation.

An assay according to the present invention can be conducted by incubating a putative PYK2 inhibitor with PYK2 pseudosubstrate and PYK2. PYK2 can be recombinant, from a natural (mammalian source), or provided in an intact or a disrupted osteoblast-like cell.

### PYK2 Pseudoenzyme

In one embodiment, recombinant PYK2 is a peptide comprising PYK2 kinase domain corresponding to PYK2 amino acid residues 414-692 (“PYK2 pseudo-enzyme”). The PYK2 pseudoenzyme can further comprise an N-terminal His-Tag. PYK2 pseudo-enzyme can be expressed in baculovirus. The PYK2 pseudo-enzyme can be purified using affinity and/or conventional chromatography.

### Optional Enhancement of PYK2 Activity

Optionally, the tyrosine kinase activity of PYK2 pseudo-enzyme (or, in other embodiments, endogenous or exogenous PYK2) can be enhanced by phosphorylating the Src phosphorylation sites (Y-579, Y-580) by incubating the PYK2 pseudo-enzyme with recombinant Src tyrosine kinase (Upstate Biochemical or similarly produced protein) and ATP using conditions recommended by the manufacturer. The phosphorylated PYK2 pseudo-enzyme is next substantially purified from Src using affinity and/or conventional chromatography.

### PYK2 Artificial Substrate

In another embodiment, PYK2 inhibitors and PYK2 inhibitor activity are identified using an exogenously added PYK2 artificial substrate such as poly (glu,tyr) [molar ratio about 4:1; Sigma Chemical Company, St. Louis, Mo.) and can be quantified as described in WO 98/35056 as follows: After osteoblast-like cells are incubated with a test PYK2 inhibitor, the cells can be solubilized in TNE lysis buffer containing 50 mM Tris-HCl (pH 7.4), mM NaCl, 1% NP-40, 1 mM EDTA, 10% glycerol, 50 mM NaF, 1 mM sodium vanadate and protease inhibitors.

Half of the sample can be subjected to immunoblotting with anti-PYK2 antibodies, and the other half can be washed 2 times with the same lysis buffer, and with kinase assay buffer (1×) containing 20 mM Tris-HCl, pH 7.4, 100 mM NaCl, 10 mM MnCl2 and 1 mM dithiothreitol. After removal of the wash buffer, 50 μl of kinase assay buffer containing 5 μCi [y-32P] ATP (3000 Ci/mmol, Amersham), 10 μM ATP, 0.1% BSA and 100 μg of poly (Glu, Tyr) can be added and incubated for 10 min at 30° C. (Howell and Cooper, 1995 Mol. Cell. Biol. 14:5402-5411). The reaction mixtures (25 μl) are added to 25 μl of 30% tri chloro acetic acid (TCA) and 0.1 M sodium pyrophosphate, followed by incubation at 4° C. for 15 min. The precipitated proteins can be transferred to a Multiscreen-FC filter plate (Millipore, Marlborough, Mass.), washed with ice cold 15% TCA (3×), allowed to dry and incorporation of ³²P into the pseudosubstrate can be counted on a Packard top count microplate scintillation counter (Packard, Meriden, Conn.).

II) The specific activity can be determined by comparing the radioactive counts with immunoblot signals. Immunoblotting can be conducted as follows: phosphotyrosine is detected by immunoblotting with HRP conjugated anti-phosphotyrosine mAb 4G10 or with anti-PYK2 polyclonal antibodies, followed by HRP-conjugated anti-rabbit IgG.

Blots can be developed by enhanced chemiluminescence (ECL, Amersham). ECL signals can be determined using an LKB uttroscan XL laser densitometer (LKB, Bromma, Sweden) and the specific activity of tyrosine phosphorylated PYK2 can be calculated by comparing the estimated phosphotyrosine contents to protein levels of PYK2. Relative specific activity of phosphorylated PYK2 is normally determined from triplicated experiments.

### PYK2-Dependant Phosphorylation Assay Using Fluorescence Polarization

In another embodiment, PYK2-dependant phosphorylation activity can be detected using fluorescence polarization. Fluorescence polarization uses a fluorescein-labeled phosphopeptide (“tracer”), a PYK2 substrate, PYK2, and optionally a putative PYK2 inhibitor. In the absence of PYK2-dependant phosphorylating activity (e.g. In the presence of a PYK2 inhibitor), a significant portion of the tracer will be bound by anti-phosphotyrosine antibody, resulting in a high polarization value. In the presence of non-inhibited PYK2-dependant phosphorylation activity, the substrate will be phosphorylated. Such phosphorylated substrate generated will compete with the tracer for binding to anti-phosphotyrosine antibodies, decreasing the amount of bound tracer and thus decreasing the fluorescence polarization value of the sample. If enough kinase reaction product is generated during the reaction, the fluorescent tracer can be completely displaced from the anti-phosphotyrosine antibodies and the emitted light will be totally depolarized. Thus, the change in fluorescence polarization is directly related to PYK2-dependant phosphorylating activity.

In another embodiment, about 150 μM of PYK2 pseudo-enzyme is Incubated with 15 μM of PYK2 pseudosubstrate in kinase assay buffer (50 mM HEPES pH 7.5, 1 mM MgCl₂, 0.1% BSA, 10 mM DTT and 50 M ATP). When the assay includes a putative PYK2 inhibitor, an appropriate vehicle is included in control incubation and ATP is added last. The reaction is allowed to proceed for 1 to 2 hours at 30° C. The reaction is stopped with the addition of a stop/detection mixture containing EDTA, 10×PTK green tracer (Invitrogen #P2843) and 10× antiphosphotyrosine antibody (Invitrogen). After 1 hour equilibration at room temperature the plates are read on Molecular Devices Analyst GT using filters and settings compatible with the green tracer. Sigmoidal dose response curves are generated using GraphPad Prism or similar software using linear regression with variable slope. When such an experiment is performed with increasing doses of PF—X, the IC50 was determined to be 30.9 nM.

In Vitro PYK2-Dependant Phosphorylation Using Cells Transformed with an Inducible PYK2.

PYK2-dependant phosphorylation can be assayed using osteoblast-like cells or osteoblast surrogate cells transformed to over-express PYK2. Constitutive PYK2 over-expression causes a number of cell types to detach from tissue culture plates over time. Optionally, PYK2-dependant phosphorylation can be assayed using cells transformed with an inducible PYK2. One skilled in the art can readily employ several inducible gene expression systems for mammalian cell culture (e.g. tetracycline, ecdysone, etc).

Optionally, cells can be transformed using the RU486 inducible system (Invitrogen). By way of example, details are given in Example 9.

III) Whereas Applicants have included subject headings in the present application, such headings are for convenience of the reader and should not be read as limitations. It should be readily obvious that many terms (by way of non-limiting example, PYK2 inhibitor, osteoblast function, osteoblast-like cell, etc.) are applicable to multiple embodiments of the present invention.

### WORKING EXAMPLES

Having now generally described the invention, the same will be more readily understood through reference to the following examples that are provided by way of illustration, and are not intended to be limiting of the present invention, unless specified.

### Example 1

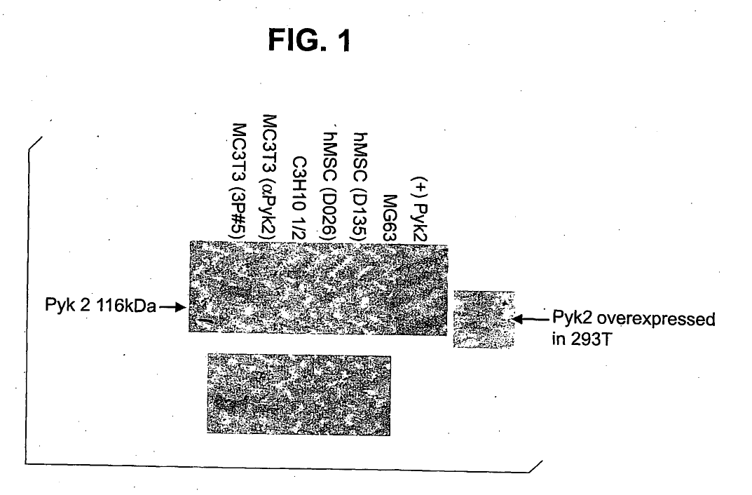

PYK2 SDS PAGE Blot. Lysates from murine (MC3T3 and C3H10T1/2) and human (mesenchymal stem cells from 2 donors and MG63) osteoblast cells were Immunoprecipitated with a polyclonal anti-PYK2 antibody (3P#5 or Santa Cruz anti-PYK2). Immunoprecipitated PYK2 was resolved by SDS-PAGE, blotted onto PVDF membranes and then probed with the anti-PYK2 polyclonal antibody followed by HRP-linked protein A. A lysate from 293T cells transfected with a PYK2 expression vector was used as a positive control. Two different exposures of the blot are shown in FIG. 1. These results demonstrate that PYK2 is expressed in murine and human osteoblast-like cells.

### Methods Used in Examples 2, 3, 4, & 6

Quantitative Alkaline Phosphatase Measurement. For quantitative alkaline phosphatase measurements, cells were washed twice with Dulbecco's phosphate buffered saline (DPBS) followed by incubation with substrate buffer (50 mM glycine, 1 mM magnesium chloride, pH 10.5) containing 1.3 mg/ml p-nitrophenol phosphate. The levels of alkaline phosphatase activity were determined by measuring the absorbance at 405 nM and compared to p-nitrophenol standards. The level of alkaline phosphatase activity was corrected for the amount of DNA in each sample.

Alkaline Phosphatase Stain (qualitative). The qualitative alkaline phosphatase was performed using a leukocyte alkaline phosphatase kit (Sigma, #85L-3R). Cells were rinsed twice with DPBS and fixed for 1 minute with citrate:acetone (2:3 vol/vol). Fixative was rinsed with DPBS and cells were stained using Fast Violet B solution containing Napthol AS-MX phosphatase alkaline solution according to the manufacturer's instructions. Cells were incubated in the dark at room temperature for one hour. Cells were rinsed three times with water.

Calcium Assay. Calcium deposited by the cells was measured using a diagnostic kit (Sigma #587A). Briefly, cells were rinsed twice with DPBS and hydrolyzed in 0.5 N HCl rotating overnight at 4°. Cells were then scraped and cellular debris was pelleted. Supernatants were used to measure calcium levels following manufacturer's protocol. The absorbance at 570 nm was determined and compared to calcium standards. Calcium levels were corrected for the DNA content in each well.

DNA Assay. DNA content is measured using Hoechst 33258 fluorescent bisbenzimide dye. Cells are washed twice with DPBS and trypsinized. Cell pellets are digested overnight at 600 using papain digestion buffer (0.1 M sodium acetate, pH 5.6, 0.05 M EDTA, 0.001 M cysteine, 150 μg/ml papain). After digestion, 100 μl of sample is added to 100 μl g/ml Hoescht dye in TNE buffer (100 mM Tris-HCl, 10 mM EDTA, 2 M NaCl, pH 7.4). Absorbance readings are measured at 356 nm/458 nm and compared to calf thymus DNA standards.

Von Kossa Staining. A Von Kossa stain was done after staining samples for alkaline phosphatase. Water was aspirated and cells were incubated with 2% silver nitrate for 10 min in the dark. Cells were then washed three times with water leaving the final rinse on the cells. Plates were placed under UV light or exposed to bright sunlight for 15 min. Cells were then rinsed three times with water and black Von Kossa nodules were photographed.

### Example 2

The role of PYK2 in osteoblast differentiation and function were studied by examining the effect of PYK2 inhibitors on alkaline phosphatase and calcium deposition by osteoblasts in vitro.

Murine mesenchymal stem cells isolated from femurs and tibiae of C57BI/6 mice were cultured in alpha-MEM containing 10% fetal bovine serum (FBS) and plated in six well dishes at a density of 3×10⁶ cells/well. The day after plating, the media were removed and replaced with media alone, in media with OS, or in OS media containing either 1 μM of dexamethasone or increasing doses of PF—Y for 21 days. “OS” medium” contains 50 μM ascorbic acid and 10 mM β-glycerophosphate. Media were refreshed every 3 days. The amount of alkaline phosphatase was measured on day 7 and day 21. The amounts of secreted calcium were determined only on day 21. The VonKossa stain was done after staining day 21 samples for alkaline phosphatase.

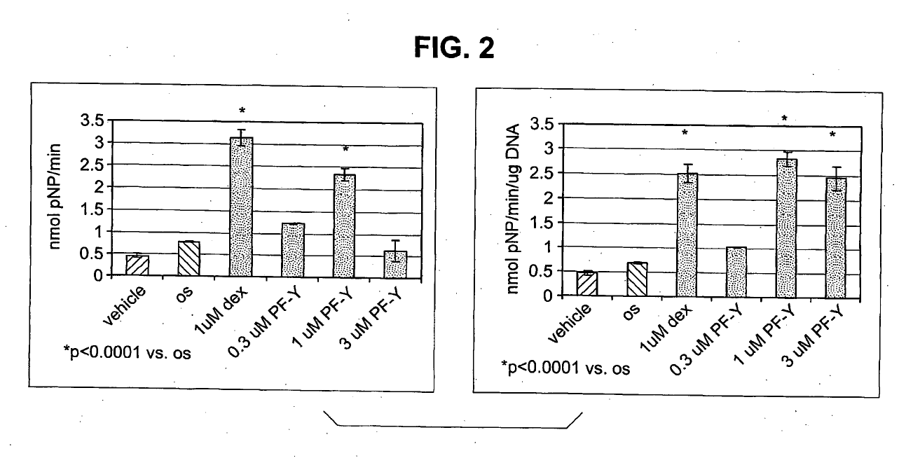

As shown in FIG. 2, incubation of murine MSCs with dexamethasone, a known agonist of osteoblast function, resulted in a stimulation of alkaline phosphatase activity (a marker of osteoblast function). PF—Y also resulted in elevated alkaline phosphatase activity. PYK2 antagonism stimulated alkaline phosphatase activity, whether expressed in units per culture (left panel) or units per μg DNA (right panel).

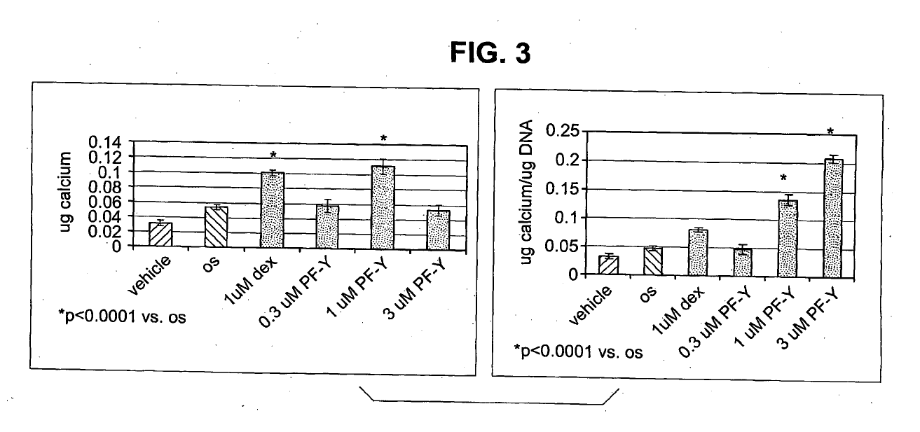

As shown in FIG. 3, incubation of murine MSCs with dexamethasone, a known agonist of osteoblast function, resulted in increased levels of calcium deposition (a marker of osteoblast function). Incubation with PF—Y also resulted in increased levels of calcium deposition. PYK2 antagonism stimulated calcium deposition, whether expressed in μg per culture (left panel) or in μg per μg DNA (right panel).

### Example 3

Human mesenchymal stem cells were cultured in DMEM-high glucose containing 10% FBS and 10 mM l-glutamine and plated in six well dishes at a density of 3×10⁴ cells/well. The day after plating, these cultures were treated and analyzed as in Example 2.

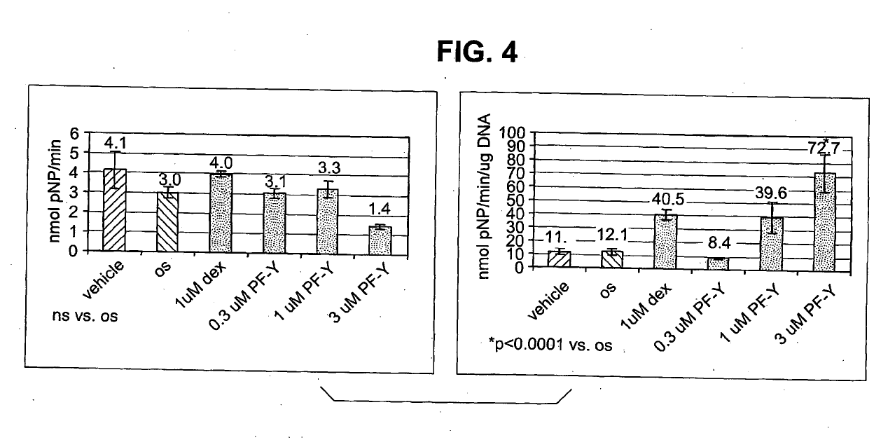

As shown in FIG. 4, incubation of human MSCs with PF—Y resulted in elevated alkaline phosphatase activity expressed in units per μg DNA (right panel).

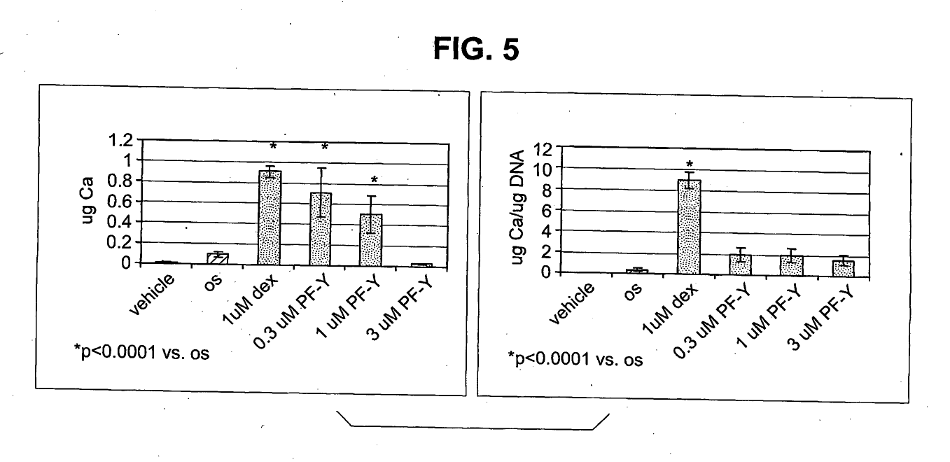

As shown in FIG. 5, incubation of human MSCs with dexamethasone, a known agonist of osteoblast function, resulted in increased levels of calcium deposition (a marker of osteoblast function). When compared to OS media alone, incubation with PF—Y (especially the two lower doses) also resulted in increased levels of calcium deposition, whether expressed in μg per culture (left panel) or in g per μg DNA (right panel).

### Example 4

MC3T3 cells were cultured in alpha-MEM media containing 10% FBS and plated in six well dishes at a density of 5×10⁴ cells/well. The day after plating, the media were removed and OS media with increasing doses of PF—Y were added. Media were refreshed every 3 days. The amount of alkaline phosphatase was measured as in Example 2 and 3.

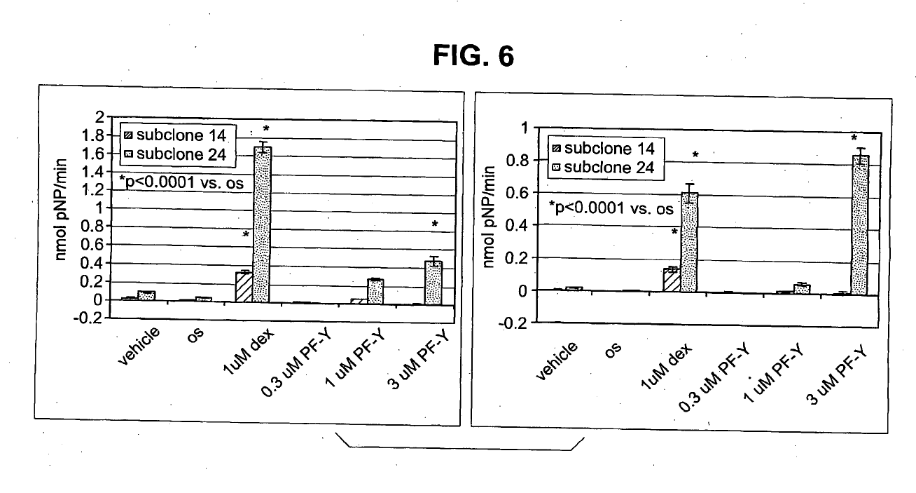

As shown in FIG. 6, incubation of murine MC3T3 cells with PF—Y resulted in elevated alkaline phosphatase activity expressed in units per plate (left panel) or units per μg DNA (right panel).

### Example 5

Murine MC3T3 cells were treated with Aluminum fluorate (AIF) alone or in the presence of 3 mM PF—Y. Cells were lysed and the amount of total PYK2 and phosphorylated PYK2 (P-Y402) were determined by immunoprecipitation with antibodies that recognize total PYK2 or phosphorylated Tyr 402 PYK2 followed by SDS-PAGE.

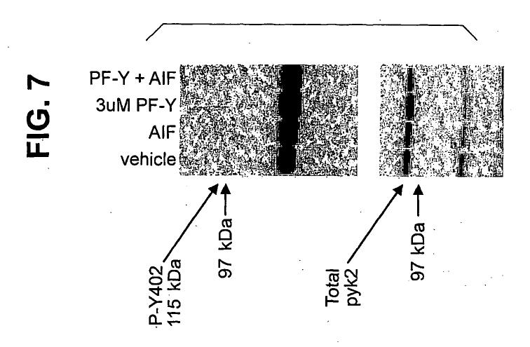

As shown in FIG. 7, AIF stimulated phosphorylation of tyrosine 402 and PF—Y inhibited AIF-induced phosphorylation.

### Example 6

PYK2 KO osteoblast assays. Bone marrow cells isolated from femurs and tibiae of C57BI/6 or PYK2 KO female mice were cultured with media alone or with media supplemented with 50 μM ascorbic acid and 10 mM β-glycerophosphate (OS) for 21 days. Media were changed every 34 days. Alkaline phosphatase activity was at day 7 and 21. The amount of calcium secreted into the extracellular matrix was measured on day 21, and extracellular matrix was visualized by the von Kossa method.

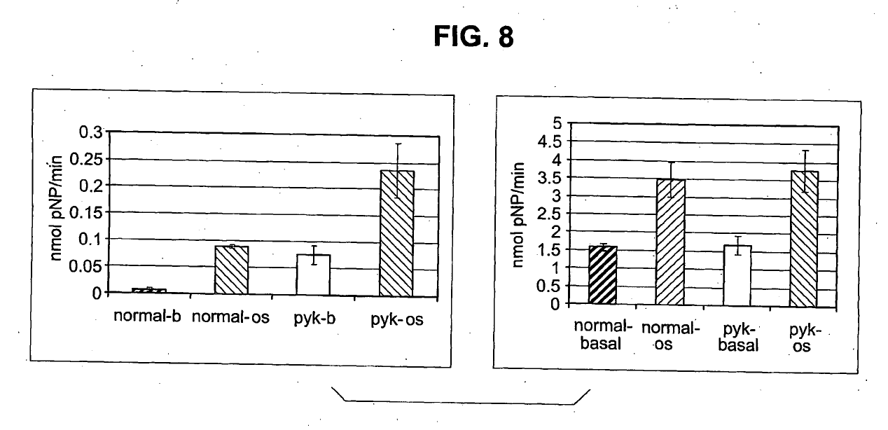

As shown in FIG. 8 (left panel), after 7 days of culture in unsupplemented media (“-basal”) or in OS media, the PYK2-deficient osteoblasts demonstrated greater alkaline phosphatase activity.

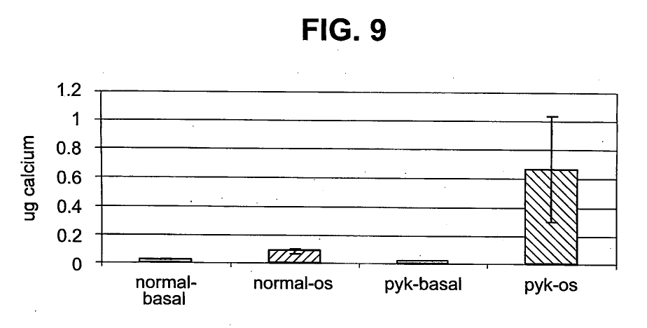

As shown in FIG. 9, extracellular calcium deposition was greatly enhanced in PYK2-deficient osteoblasts cultured in OS medium when compared to wild-type osteoblasts.

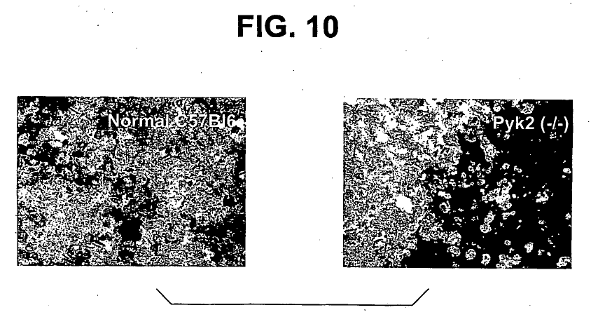

As shown in FIG. 10, extracellular calcium deposition was greatly enhanced in PYK2-deficient osteoblasts cultured in OS medium when compared to wild-type osteoblasts as visualized by Von Kossa stain.

### Example 7

Pyk2 knockout mice: Pyk2 knockout mice were developed as described in Okigaki et al., PNAS, 100(19):10740-10745, 2003.

Female Pyk2 knockout mice (n=7) and female wild-type littermate (C57BI/6) controls (n=5) at 6 months of age were subcutaneously injected with tetracycline (20 mg/kg) on 10 days and with calcein (20 mg/kg) on 4 days before sacrifice as fluorescent bone markers for determination of bone formation. Micro-computerized tomography (Scanco micro-CT 40, Scanco Medical AG, Bassersdorf, Switzerland) analysis of distal femoral metaphysis and the 4^{th} lumbar vertebral was performed to evaluate the change in bone mass and bone structures. Static and dynamic histomorphometric measurements were perform on undecalcified longitudinal sections of the 4^{th} lumbar vertebral bodies. Further, bone strength was evaluated using a four-point-bending test at the femoral shaft.

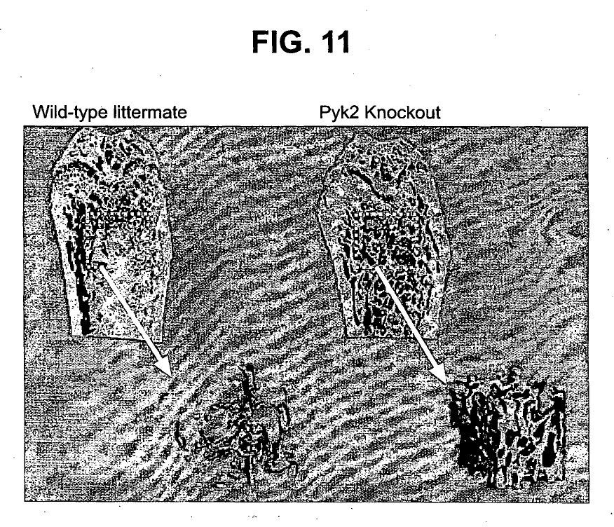

Micro-computerized tomography analysis of distal femoral metaphysis showed a significant increase in female Pyk2 knockout mice compared with female wild-type littermate (C57BI/6) controls at 6 months of age (FIG. 11).

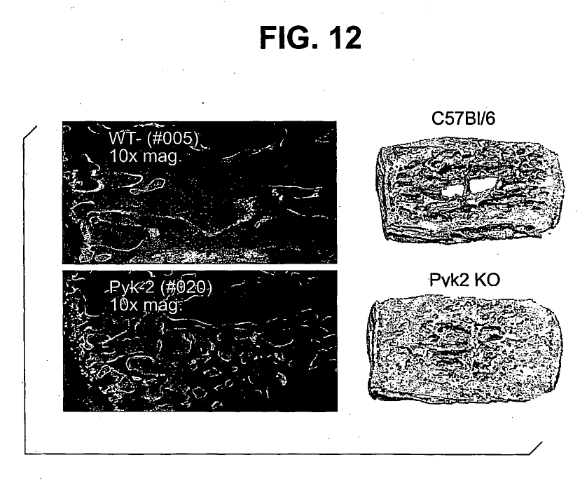

Similarly, micro-computerized tomography analysis of the 4^{th} lumbar vertebral body showed a significant increase in female Pyk2 knockout mice compared with female wild-type littermate (C57BI/6) controls at 6 months of age as seen in FIG. 12 right panel.

Compared with female wild-type littermate (C57BI/6) controls, female Pyk2 knockout mice showed statistically significant increases in trabecular bone volume, trabecular thickness and trabecular number (ranges from +48% to +206%) and decrease in trabecular separation (−67%). Dynamic histomorphometric analysis showed Pyk2 knockout mice had significantly elevated bone formation that includes statistically significant increases (ranges from +22% to +323%) in percent mineralizing surface (MS/BS), mineral apposition rate (MAR), bone formation rate-surface referent (BFR/BS) and bone formation-tissue volume referent (BFR/TV) compared with female wild-type littermate (C57BI/6) controls. FIG. 12 left panel illustrated that Pyk2 knockout mice (bottom) had significantly more fluorescent labels on bone surface, indicating increased bone mineralization and bone formation, as compared with wild-type littermate (C57BI/6) control (top). Femurs from Pyk2 knockout mice were significantly stiffer and required significantly greater load to break compared with wild-type littermate (C57BI/6) controls.

In conclusion, these data demonstrate that a deficiency in Pyk2 leads to an increase in bone formation, bone mass and bone strength.

### Example 8

The effect of treatment of a mammal with a PYK2 antagonist of Formula I, namely the di-hydrochloride salt of PF—X, was examined. PF—X is a PYK2 inhibitor with an IC50 of 30.9 nM. The ovariectomized (OVX) rat model was used.

Animal and study design: Fifty 5-month-old Sprague-Dawley female rats (Taconic Farms Inc, German Town, N.Y.), weighing approximately 330 grams and at 4.5-5 month old, were used in this study. The animals were housed at 24° C. with a 12 h light/12 h dark cycle and allowed free access to water and a commercial diet (Purina laboratory Rodent Chow 5001, Purina-Mills, St. Louis, Mo.) containing 0.95% calcium, 0.67% phosphorus, and 4.5 IU/g vitamin D₃. The experiments were conducted according to Pfizer Animal Care and Use approved protocols and animals were maintained in accordance with the ILAR (Institute of Laboratory Animal Research) Guide for the Care and Use of Laboratory Animals. Ten rats were sham-operated (sham) and treated by daily oral gavage with vehicle (20% beta-cyclodextrin, 1 ml/rat), while the remaining rats (n=10/group) were bilaterally ovariectomized (OVX) and treated by oral gavage with either vehicle, PF-Xat doses of 10 or 30 mg/kg/d, or 17□-ethynyl estradiol (EE) at 30 μg/kg/d for 28 days beginning 1 day post-surgery. All rats were given subcutaneous injections of 10 mg/kg calcein (Sigma Chemical Co., St. Louis, Mo.), a fluorochrome bone marker, at 12 and 2 days before sacrifice in order to determine dynamic changes in bone tissues (Frost H M 1969 Tetracycline-based histologic analysis of bone remodeling. Calcif Tissue Int 3:211-237). After 4 weeks of treatment, the rats were weighed, and body weight gain was obtained. Next the rats were euthanized by cardiac puncture under ketamine/xylazine anesthesia.

Serum osteocalcin: Serum was obtained by tall bleeding after 2 weeks of treatment Serum osteocalcin was determined by RIA (Price P A, Nishimoto S K 1980 Radioimmunoassay for the vitamin K-dependent protein of bone and its discovery in plasma. Proc Natl Acad Sci USA 77: 2234-2238).

Peripheral Quantitative Computerized Tomography (pQCT) Analysis: Excised femurs were scanned by a pQCT X-ray machine (Stratec XCT Research M, Norland Medical Systems, Fort Atkinson, Wis.) with software version 5.40. A 1-mm thick cross section of the femur metaphysis was taken at 5.0 mm proximal from the distal end with a voxel size of 0.10 mm. Cortical bone was defined and analyzed using contour mode 2 and cortical mode 4. An outer threshold setting of 340 mg/cm³ was used to distinguish the cortical shell from soft tissue and an inner threshold of 529 mg/cm³ to distinguish cortical bone along the endocortical surface. Trabecular bone was determined using peel mode 4 with a threshold setting of 655 mg/cm³ to distinguish (sub)cortical from cancellous bone. An additional concentric peel of 1% of the defined cancellous bone was used to ensure (sub)cortical bone was eliminated from the analysis. Volumetric content, density, and area were determined for both trabecular and cortical bone. Using the above setting, we have determined that the ex vivo precision of volumetric content, density and area of total bone, trabecular, and cortical regions ranged from 0.99% to 3.49% with repositioning (Ke H Z et al., Lasofoxifene. a selective estrogen receptor modulator, prevents bone loss induced by aging and orchidectomy in the adult rat. Endocrinology, 141:1338-1344, 2000).

Proximal Tibial Metaphyseal (PTM) Trabecular Bone Histomorphometry: At necropsy, the proximal third of the right tibia from each rat was removed, dissected free of soft tissue, fixed in 70% ethanol, stained in Villanueva bone stain, dehydrated in graded concentrations of ethanol, defatted in acetone, and embedded in methyl methacrylate. Longitudinal sections of proximal tibial metaphysis at 4 and 10 μm thickness were prepared for histomorphometry as described previously (Baron R, Vignery A, Neff L, Silvergate A, Maria AS 1983 Processing of undecalcified bone specimens for bone histomorphometry. In: Recker RR, ed. Bone Histomorphometry: Techniques and Interpretation. Boca Raton, Fla.: CRC Press, 13-36.

Additional methodology was reported in Jee W S S, Li X J, Inoue J, Jee K W, Haba T, Ke H Z, Setterberg R B, Ma Y F 1997 Histomorphometric assay of the growing long bone. In: Takahashi H., ad. Handbook of Bone Morphology. Nishimusa, Niigata City, Japan, 87-112).

Trabecular bone histomorphometric analysis was performed using an Image Analysis System (Osteomeasure, Inc., Atlanta, Ga.). Histomorphometric measurements were performed in trabecular bone tissue of the proximal tibial metaphyses between 0.5 mm and 3.5 mm distal to the growth plate-epiphyseal junction, and extended to the endocortical surface in the lateral dimension.

Measurements and calculations related to trabecular bone volume and structure included trabecular bone volume (TBV), thickness (Tb.Th), number (Tb.N), and separation (Tb.Sp), while measurements and calculations related to bone resorption included osteoclast surface and osteoclast number.

The parameters related to bone formation included percent mineralizing surface [(double labeling surface+½ single labeling surface)/total trabecular surface×100], mineral apposition rate, bone formation rate/TV, bone formation rate/BV, bone formation rate/BS.

The definitions and formulae for calculations of these parameters are described previously by Parfitt et al. (Parfitt A M, Drezner M K, Glorieux F H, Kanis J A, Malluche H, Meunier P J, Ott S M, Recker R R 1987 Bone histomorphometry: Standardization of nomenclature, symbols, and units. J Bone Miner Res 2:595-610).

Additional methodology is described in Jee et al. (Jee W S S, Li X J, Inoue J, Jee K W, Haba T, Ke H Z, Setterberg R B, Ma Y F 1997 Histomorphometric assay of the growing long bone. In: Takahashi H., ed. Handbook of Bone Morphology. Nishimusa, Niigata City, Japan, 87-112).

Study results and discussion: OVX rats treated with vehicle increased significantly body weight compared with sham controls. OVX rats treated with EE prevented OVX-induced weight gain. No significant difference in body weight between OVX rats treated with vehicle or PF—X at both doses.

Serum osteocalcin, a bone formation marker, was significantly increased in PF—X-treated OVX rats while it was significantly decreased in EE-treated OVX rats compared with vehicle-treated OVX rats at 2 weeks post-treatment. These data indicate that EE decreased while a PYK2 inhibitor increased bone formation in OVX rat model of human osteoporosis.

PQCT analysis of distal femoral metaphysis showed that there was significant increases in total bone mineral content, total bone mineral density, total bone area, trabecular bone density and cortical bone content in 10 or 30 mg/kg/d of PF—X treated OVX rats compared with vehicle treated OVX rats, indicating that a PYK2 inhibitor increases both trabecular and cortical bone in OVX rat model. EE-treated OVX rats had higher total bone mineral content, total bone mineral density, and cortical bone content compared with vehicle treated OVX rats.

Trabecular bone histomorphometric analysis of proximal tibial metaphysis showed that there was a significant increase in trabecular bone volume, trabecular thickness, mineral apposition rate, percent mineralizing surface, bone formation rate/BV and bone formation rate/TV, and a significant decrease in osteoclast surface and osteoclast number in 30 mg/kg/d of PF—X treated OVX rats compared with vehicle-treated OVX rats. These data indicate a PYK2 inhibitor increases bone mass by a combination of increasing osteoblast number and osteoblast activity. In contrast, EE treatment in OVX rats decreases mineral apposition rate, percent mineralizing surface, bone formation rate/BV and bone formation rate/TV, osteoclast surface and osteoclast number.

These data demonstrate that PF—X, a PYK2 inhibitor, stimulates osteoblast function and number.

### Example 9

The full-length human PYK2 cDNA was cloned into pGENE containing a V5-His epitope Tag. (Invitrogen). This plasmid was transfected into NIH 3T3 Switch cell line (Invitrogen) and clonal lines were selected in the appropriate selection media and Isolated using cloning cylinders. Clones were analyzed for inducible PYK2 gene expression using cell lysates and Western Blot and detection with anti-PYK2 or anti-V5 epitope Tag antibodies.

PF—X was analyzed for PYK2 inhibition as follows: the selected GeneSwitch PYK clonal line was plated in growth media (DMEM high glucose supplemented with 10% Calf Serum, 1× glutamine, 50 μg/ml Hygromycin, 150 μg/ml Zeocin (all cell culture products are obtained from Invitrogen) into Biocoat® collagen-coated plates (Becton-Dickinson catalog #359132). The following day the medium was changed to serum free. The following day, pyk2 expression was induced with 10 nM final mifepristone (RU486). After a 4 hour induction period, test compound or vehicle was added to the appropriate wells. After a one hour treatment period, the cells were fixed by replacing the medium with freshly diluted formaldehyde in PBS (1:10 of 37% solution) for 20 minutes at room temperature. Cells were then permeabilized with 4×100 μl washes (5 minutes each, with rotary shaking) of 0.1% Triton X-100 in PBS at room temperature. Nonspecific binding was prevented by blocking overnight at 4° C. with 100 μl Odyssey blocking buffer (licor.com catalog #927-40000).

The following day, primary antibody (pyk2 phospho-Y402, Biosource catalog #44-618G) was added at 1:200 in Odyssey Block for 2 hours with rotary shaking at room temperature. Alternatively, depending on the cell-type, antibodies to other PYK2 phospho-substrates may be substituted (e.g. cortactin phospho-Y421, Sigma C0739; paxillin phospho-Y31 Sigma P6368). After 4×5 minute washes with PBS Tween 20, 0.1%, IR Dye 800-conjugated anti-rabbit secondary antibody (Rockland catalog #611-132-122) was added for 1 hour at room temperature with rotary shaking. After the same washing regimen, plates were blotted dry and scanned in the LICOR instrument. To determine the IC50, the relative signal of the PF—X treated group was compared to that of vehicle using curve fitting software (e.g. GraphPad Prism, linear regression with variable slope). Using this procedure, PF—X was found to have an IC50 of about 136 nM.

What is claimed is:

1. A method of stimulating osteoblast function in a mammal comprising administering a PYK2 inhibitor to a mammal in need thereof in an amount effective to stimulate an osteoblast function.

2. The method of claim 1 wherein the PYK2 inhibitor is a trifluoromethylpyrimidine compound.

3. The method of claim 1 wherein the PYK2 inhibitor is a 5-aminooxindole compound.

4. The method of claim 1 wherein the PYK2 inhibitor is a tertiary aminopyrimidine compound.

5. The method of claim 1 wherein the PYK2 inhibitor is the compound of formula PF—X.

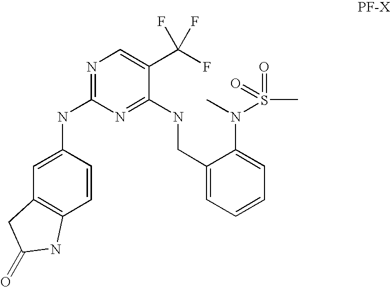

6. The method of claim 1 wherein the PYK2 inhibitor is the compound of formula PF—Y.

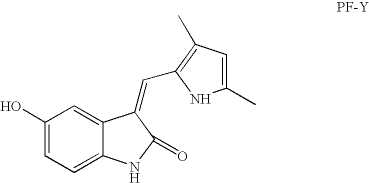

7. The method of any one of claims 1-6 wherein the PYK2 inhibitor is a selective PYK2 inhibitor.

8. The method of any one of claims 1-6 wherein the PYK2 inhibitor is a FAK inhibitor.

9. The method of claim 1 wherein the PYK2 inhibitor is a Flk inhibitor.

10. The method of any one of claims 1-6 wherein the PYK2 inhibitor is a direct PYK2 inhibitor.

11. The method of any one of claims 1-6 wherein the mammal has osteoporosis, osteopenia, bone fracture, osteomalacia, rickets, fibrogenesis imperfecta ossium, or low bone density or risk thereof.

12. The method of any one of claims 1-6 wherein the mammal has childhood idiopathic bone loss or periodontitis bone loss.

13. The method of claim 11 wherein the osteoporosis is glucocorticoid-induced osteoporosis, hyperthyroidism-induced osteoporosis, immobilization-induced osteoporosis, heparin-induced osteoporosis, post-menopausal osteoporosis, vitamin D deficient osteoporosis, or immunosuppressive-induced osteoporosis.

14. The method of any one of claims 1-6 wherein the mammal is human.

15. The method of any one of claims 1-6 wherein the osteoblast function is osteoid production, mineralization, osteopontin production, osteonectin production, extracellular calcium accumulation, or bone healing.

16. The method of any one of claims 1-6 wherein the mammal is in need of bone healing.

17. The method of any one of claims 1-6 wherein the mammal is in need of bone healing following facial reconstruction, maxillary reconstruction, mandibular reconstruction, vertebral synostosis, bone graft, osteotomy, or prosthetic implantation.

18. The method of any one of claims 1-6 further comprising administration of an amount of a second therapeutic bone agent.

19. The method of claim 18 wherein the second therapeutic bone agent is a bone anabolic agent, an anti-resorptive agent, or an anabolic anti-resorptive agent.

20. The method of claim 18 wherein the second therapeutic bone agent is (−)cis-6-phenyl-5-[4-(2-pyrrolodin-1-yl-ethoxy)-phenyl]-5,6,7,8-tetrahydronapthalen-2-ol or a pharmaceutically acceptable salt thereof.

21. The method of claim 18 wherein the second therapeutic bone agent is a PGE2 EP2 selective receptor agonist.
